(19) United States
(12) Patent Application Publication
(10) Pub. No.: US 2017/0343521 A1
(43) Pub. Date: Nov. 30, 2017

(54) APPARATUS AND METHOD FOR GENERATING OLFACTORY INFORMATION

(71) Applicant: ELECTRONICS AND TELECOMMUNICATIONS RESEARCH INSTITUTE, Daejeon (KR)

(72) Inventors: Sung-June CHANG, Daejeon (KR); Hae-Ryong LEE, Daejeon (KR); Jun-Seok PARK, Daejeon (KR); Jong-Woo CHOI, Daejeon (KR); Hyung-Gi BYUN, Samcheok-si (KR); Jang-Sik CHOI, Samcheok-si (KR)

(21) Appl. No.: 15/408,543

(22) Filed: Jan. 18, 2017

(30) Foreign Application Priority Data
May 26, 2016 (KR) 10-2016-0064918
Jan. 3, 2017 (KR) 10-2017-0000870

(51) Int. Cl. G01N 33/00; G01N 9/36

(52) U.S. Cl. CPC G01N 33/0001; G01N 9/36; G01N 33/0027

(57) ABSTRACT

An apparatus and method for generating olfactory information. The apparatus for generating olfactory information generates olfactory information that can be shared between a real world and at least one virtual world. The apparatus for generating olfactory information includes a sensor and a processor. The sensor recognizes a real-world odor, and acquires the original data of the result of the detection of the real-world odor. The processor acquires representative data including the evaluation of the quantitative numerical value of the real-world odor by analyzing the original data, and generates real-world olfactory information including both the original data and the representative data.

### CROSS-REFERENCE TO RELATED APPLICATIONS

This application claims under 35 U.S.C. §119(a) the benefit of Korean Application Nos. 10-2016-0064918 and 10-2017-0000870, filed on May 26, 2016 and Jan. 3, 2017, respectively, which are incorporated herein by references.

### BACKGROUND OF THE INVENTION

### 1. Field of the Invention

The present invention generally relates to the capability representation of electronic nose equipment and a representation method for the conveyance of a perceived odor in an Moving Picture Experts Group (MPEG)-V-based virtual reality system, and more particularly to MPEG-V (Media Context and Control) technology for providing intercompapatibility between a virtual world and the real world in a virtual reality system.

### 2. Description of Related Art

Electronic noses (e-noses) are being used as sensors that detect particles or gases that cause odors in the real world. In the real world, odors are each detected based on the density of a gas or the density of particles causing the odor by using a physical, chemical or biological method.

In MPEG-V standardization conferences, attempts have been made to standardize a method for represent olfactory information, detected by e-nose sensors, for the purpose of reconstructing the olfactory information in a virtual world or the real world.

Accordingly, there is an urgent need for the development of data types for the sharing of olfactory information between a virtual world and the real world that has been advanced and standardized in the MPEG-V standardization conferences, as described above.

### SUMMARY OF THE DISCLOSURE

An object of the present invention is to provide intercompapatibility between a virtual world and the real world by recognizing odors present in the real world within the scope of MPEG-V and then conveying the real-world odors to the virtual world.

An object of the present invention is to generate and convey detailed information in a process of conveying a real-world odor to a virtual world. That is, the present invention is intended to convey the density of a gas, i.e., the cause of a real-world odor, to a virtual world without a change to raw data and to also convey a quantitative evaluation result based on a human organoleptic test to the virtual world, thereby more faithfully reconstructing a mood related to the real-world odor in the virtual world.

An object of the present invention is to map a change in the density of a gas in the real-world to a human-based quantitative evaluation result by taking into account the olfactory adaptation effect over time, thereby providing a means for reducing the fatigue of olfaction and effectively reconstructing real-world olfactory information in a virtual world.

An object of the present invention is to provide a method and apparatus for generating olfactory information, which are capable of effectively dealing with harmful gases present in the real world. A full preparation for the safety management of harmfulness gases that cannot be perceived by humans can be made in such a way as to map the strength of an odor, perceived by humans for each specific gas, to a quantitative evaluation result for the harmfulness of the gas.

In order to achieve one or more of the above objects, a method for generating olfactory information according to an embodiment of the present invention assumes a case where both the density of a chemical detected by an e-nose and the strength perceived by humans are recorded. The information evaluated as a quantitative numerical value perceived by human olfaction is generated as information having a format, such as an XML format.

According to an aspect of the present invention, there is provided a method for generating olfactory information, which generates olfactory information that can be shared between the real world and at least one virtual world, the method including: acquiring, by a sensor capable of detecting a real-world odor, the original data of the result of the detection of the real-world odor, i.e., quantitative and qualitative information for an actually detected gas; acquiring, by an analysis processor for the original data, representative data including the evaluation of the quantitative numerical value of the real-world odor; and generating real-world olfactory information including both the original data and the representative data.

The original data may be quantitative and qualitative information for an actually detected gas, and the representative data may refer to information obtained through the evaluation of a strength perceived by human olfaction.

The real-world olfactory information including the original data and the representative data may have a format, such as an XML format, and may be generated as information in a form having compatibility in various platforms.

The representative data may include a density interval quantitatively representative of an imperceptible case that cannot be perceived by humans.

Each of the original data and the representative data may include values over time. For example, the original data may include the series of results of the detection of the real-world odor over time, and the representative data may include the series of evaluations of the quantitative numerical values of the real-world odor over time. The elapse of time may be represented by timestamps.

A method for generating olfactory information according to another embodiment of the present invention is configured to represent the harmfulness of a material detected by an e-nose as a quantitative index having a format, such as an XML format.

According to another aspect of the present invention, there is provided a method for generating olfactory information, which generates olfactory information that can be shared between the real world and at least one virtual world, the method including: acquiring by a sensor capable of detecting a real-world odor, the original data of the result of the detection of the real-world odor, i.e., quantitative and qualitative information for an actually detected gas; acquiring, by an analysis processor for the original data, representative data, i.e., harmfulness information including the quantitative evaluation of the harmfulness of the real-world odor; and generating real-world olfactory information including the representative data.

The present invention may be configured to map a quantitative evaluation result for the harmfulness of each specific gas to an actual gas density for the strength of an odor sensed by humans when they perceive the specific gas and to then monitor the harmfulness of harmful gases and the influence of the harmful gases on human bodies.

When only original olfaction data is provided without representative data, only information about the density of an actual gas is provided to a virtual world. Accordingly, there is no information about how virtual characters react to a gas in the virtual world, and thus there occurs a compatibility error in which the virtual characters unrealistically react to the gas. Since such virtual characters are objects with which actual users chiefly commune or have empathy, the reliability of the virtual world rapidly becomes degraded due to such a compatibility error, with the result that a great limitation may be imposed on the range of use of the virtual world. Accordingly, the provision of reliable representative data information is significantly important. The present invention is configured to generate olfactory information by associating representative data with original data and to convey the olfactory information, thereby improving reliability in the utilization of the olfactory information in a virtual world.

As described above, representative data is essential to a virtual world. The generation of representative data requires millisecond-level fine olfaction original data over a long period of time. Since the amount of data is considerably large, the direct transmission of original data is ultimately a burdensome selection. The conversion of olfactory information into representative data processed to some extent, which is performed to convey olfactory information to a virtual world, will be an inevitable selection for the evolution of a virtual world. In this case, the appropriate-level provision of original data in connection with the generation and transfer of representative data is a means for significantly increasing the sensation of reality during reconstruction in a virtual world while effectively reducing the amount of data to be transmitted.

### BRIEF DESCRIPTION OF THE DRAWINGS

The above and other objects, features and advantages of the present invention will be more clearly understood from the following detailed description taken in conjunction with the accompanying drawings, in which:

FIG. 1 is an operation flowchart showing a method for generating olfactory information, which generates representative data, including the original data of a gas density and the quantitative evaluation of a real-world odor, according to an embodiment of the present invention;

FIG. 2 is an operation flowchart showing a method for generating olfactory information, which generates representative data, including the quantitative evaluation of the harmfulness of a real-world odor, according another embodiment of the present invention;

FIG. 3 is a diagram showing an apparatus for generating olfactory information according to an embodiment of the present invention;

FIG. 4 is a diagram showing correlations between the strengths (gas densities) of real-world odors and quantitative evaluation indices perceived by humans according to an embodiment of the present invention;

FIG. 5 is a diagram showing correlations between changes in the strength of a real-world odor over time and quantitative evaluation indices perceived by humans over time according to an embodiment of the present invention;

FIGS. 6 and 7 are diagrams showing the representative data of quantitative evaluation indices over time according to embodiments of the present invention;

FIG. 8 is a diagram showing correlations between the strengths (gas densities) of real-world odors, quantitative evaluation indices perceived by humans, and evaluation indices for harmfulness according to an embodiment of the present invention;

FIG. 9 is a diagram showing the XML representation syntax of Sensed Information Base Type according to an embodiment of the present invention;

FIG. 10 is a diagram showing the binary representation syntax of SensedInfoBaseType according to an embodiment of the present invention;

FIG. 11 is a diagram showing the semantics of SensedInfoListType according to an embodiment of the present invention;

FIG. 12 is a diagram showing the binary representation syntax of the strengths of odors according to an embodiment of the present invention;

FIG. 13A is a diagram showing the binary representation syntax of the strengths of odors;

FIG. 13B is a diagram showing the modified strength binary representation syntax of the strengths of odors in which an imperceptible step has been added to the binary representations of the strengths of odors according to an embodiment of the present invention;

FIG. 14 is a diagram schematically showing a process of generating information about the generation of a hazardous odor when the hazardous odor is generated during a process of generating olfactory information according to an embodiment of the present invention;

FIGS. 15A and 15B are diagrams showing the XML representation syntax of E-Nose Sensor Type according to an embodiment of the present invention;

FIG. 16 is a diagram showing the binary representation syntax of Enose Sensor Type according to an embodiment of the present invention;

FIGS. 17, 18A, 18B, and 19 are diagrams showing the semantics of EnoseSensorType according to an embodiment of the present invention; and

FIG. 20 is a diagram showing a syntax designed to define harmfulness in the XML representation syntax of E-Nose Sensor Type according to an embodiment of the present invention.

### DETAILED DESCRIPTION OF THE DISCLOSURE

Embodiments of the present invention will be described in detail with reference to the accompanying drawings. In the following description of the present invention, a detailed description of a related well-known component or function will be omitted when it is determined that the detailed description may make the gist of the present invention obscure. Furthermore, for ease of description, part of the embodiments shown in the drawings may be illustrated as being exaggerated.

The prevent invention is not limited to the embodiments. Throughout the accompanying drawings, the same reference symbols designate the same components.

A general virtual world processing system included as part of the configuration of the present invention may correspond to an engine, a virtual world, and the real world. The real world may include e-nose equipment configured to detect information about the real world, or an odor generation device configured to implement information about a virtual world in the real world. Furthermore, the virtual world may include the virtual world itself adapted to be implemented by a program, or an odor generation media reconstruction device configured to reconstruct content including odor generation information that can be implemented in the real world.

For example, the e-nose equipment may detect a real-world odor and information about the capability, specifications and the like of the e-nose equipment, and may transmit the detected information to the engine. The e-nose equipment may include Enose Capability Type adapted to transfer the capability and specifications of the e-nose equipment to the engine, Odor Sensor Technology CS adapted to describe the type of sensor required to define Enose Capability Type, and Enose Sensed Info Type adapted to transfer information, detected by the e-nose equipment, to the engine.

The engine may transmit detected information to the virtual world. In this case, an effect corresponding to Enose Sensed Info Type corresponding to a real-world odor may be implemented in the virtual world by applying the detected information virtual world.

An effect event occurring in the virtual world may be operated by the real-world odor generation device. A sensory effect, i.e., information about the effect event occurring in the virtual world, may be transmitted to the engine. Furthermore, virtual world object characteristics may be exchanged between the virtual world and the engine.

The odor generation device present in the real world and the provision of a preference of a user are described within the scope of MPEG-V. The odor generation device is present in the real world, and provides an odor to the user, thereby functioning to enable synchronization to be performed in connection with content in the virtual world and also enable the sensation of reality to be felt. For this purpose, Scent Capability Type is defined to transfer the capability and specifications of the odor generation device to the engine. Furthermore, Scent Preference Type is defined to provide a preference of the user in order to compensate for a difference between the characteristics of the odor provided by the odor generation device and the characteristics of the odor perceived by the user. Furthermore, Scent Effect is defined to describe a command for the odor generation device to generate an odor.

A generalized virtual world processing method included as part of the configuration of the present invention may be implemented by exchanging olfactory information related to the virtual world, the real world and another virtual world between the virtual world and the real world or between the virtual world and the other virtual world and then representing the olfactory information via the odor generation device. The generalized virtual world processing method may be configured to acquire virtual information, i.e., olfactory information in the virtual world, to acquire real information, i.e., real-world olfactory information, via a reality recognition unit configured to recognize an odor, to provide the virtual information to the real world or other virtual world, to provide the real information to the virtual world or other virtual world, and to provide an odor to a user based on the virtual information and the real information via an odor generation device.

The real information includes Enose Capability Type adapted to transfer the capability and specifications of the e-nose equipment, i.e., the reality recognition unit, to an engine, Odor Sensor Technology CS adapted to describe the type of sensor required to define Enose Capability Type, and Enose Sensed Info Type adapted to transfer information, detected by the e-nose equipment.

Furthermore, the method includes the steps of defining Scent Capability Type adapted to transfer the capability and specifications of the odor generation device to the engine, defining Scent Preference Type adapted to provide a preference of the user in order to compensate for a difference between the characteristics of the odor provided by the odor generation device and the characteristics of the odor perceived by the user, and defining Scent Effect adapted to describe a command for the odor generation device to generate an odor.

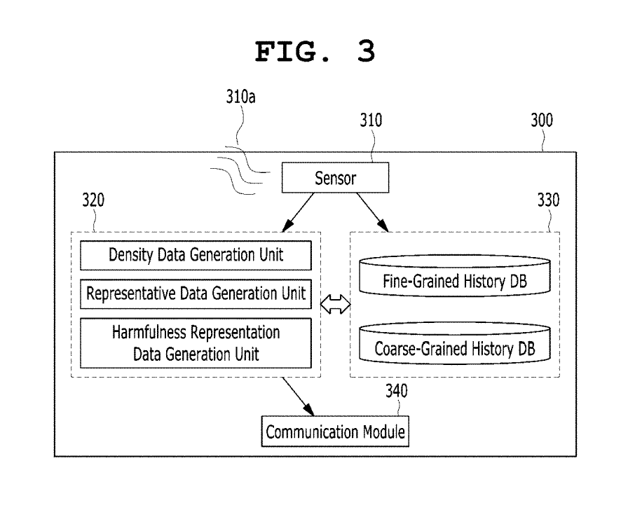

FIG. 3 is a diagram showing an apparatus 300 for generating olfactory information according to an embodiment of the present invention.

Although the apparatus 300 for generating olfactory information shown in FIG. 3 may be implemented in the form of an e-nose, it may be installed in conjunction with a gas sensor. The apparatus 300 for generating olfactory information includes a sensor 310, a processor 320, a database 330, and a communication module 340.

Although not explicitly shown in FIG. 3, a user interface, such as a button or a switch configured to enable a command to turn on or off power to be input from the outside, may be further included. Furthermore, a user interface, such as a keypad, a touch screen, a microphone or the like, configured to receive a simple operation command may be further included.

The sensor 310 recognizes a real-world odor. A flow 310a of gas particles constituting the real-world odor is detected by the sensor 310. In this case, the sensor 310 may be implemented as a combination of a plurality of gas sensors that detect a specific type of gas. A plurality of gas sensors may detect different types of gases. For the same type of gas, a plurality of gas sensors corresponding to different density ranges may be included.

The sensor 310 acquires the result of the detection of the real-world odor as original data. The original data includes quantitative and qualitative information about the actually detected gas. The quantitative information is the density of the gas or the densities of the gas over time. The qualitative information may include the type of gas and information about a situation in which the gas was detected. This operation may be more accurately performed by a density data generation unit present inside the processor 320.

The processor 320 may convert the original data of the real-world odor into representative data including the evaluation of the quantitative numerical value of the real-world odor. In this case, the quantitative evaluation may be performed by using the information of an organoleptic test based on the perception of the odor by humans in the real world. The information of the organoleptic test of each gas may be stored in the database 330. A representative data generation unit present inside the processor 320 may perform conversion into the representative data.

The processor 320 generates real-world olfactory information including both the original data and the representative data. The representative data includes information about a threshold value, i.e., a lower limit at which humans can perceive a specific gas. In other words, the representative data may include information about a gas density range that is imperceptible to a human.

The sensor 310 may track a gas density over time, and may store the gas density, together with a timestamp, in the database 330. The original data generated by the sensor 310 over time may be transferred to the processor 320 via the database 330. The processor 320 may convert the original data over time into quantitative evaluation information according to a gas density range. The processor 320 may generate the quantitative evaluation information over time as representative data.

A harmfulness representation data generation unit inside the processor 320 may generate harmfulness representation data, including quantitative evaluation information about harmfulness, which corresponds to the original data.

The communication module 340 may transfer both the representative generated by the data processor 320 and the original data generated by the sensor 310 to the external server or relay device. The processor 320 may generate XML-type olfactory information including both the original data and the representative data. The communication module 340 may replace the transfer of the original data and the representative data with the transfer of the XML-type olfactory information.

Although the communication module 340 may transfer generated olfactory information to the outside in real time, generated olfactory information may be stored in the database 330 and then transferred to the outside by the communication module 340 at predetermined time intervals. In this case, for ease of description, the result information of the collection of olfactory information generated in real time may be referred to as a “fine-grained history.” Result information in which fine-grained histories have been accumulated may be referred to as a “coarse-grained history.” A coarse-grained history is the result of the accumulation and analysis of fine-grained histories over a long period of time, and may include the trend of changes in sensor information over a long period of time, which cannot be identified from a fine-grained history. A fine-grained history is stored in a fine-grained history DB inside the database 330, and a coarse-grained history is stored in a coarse-grained history DB.

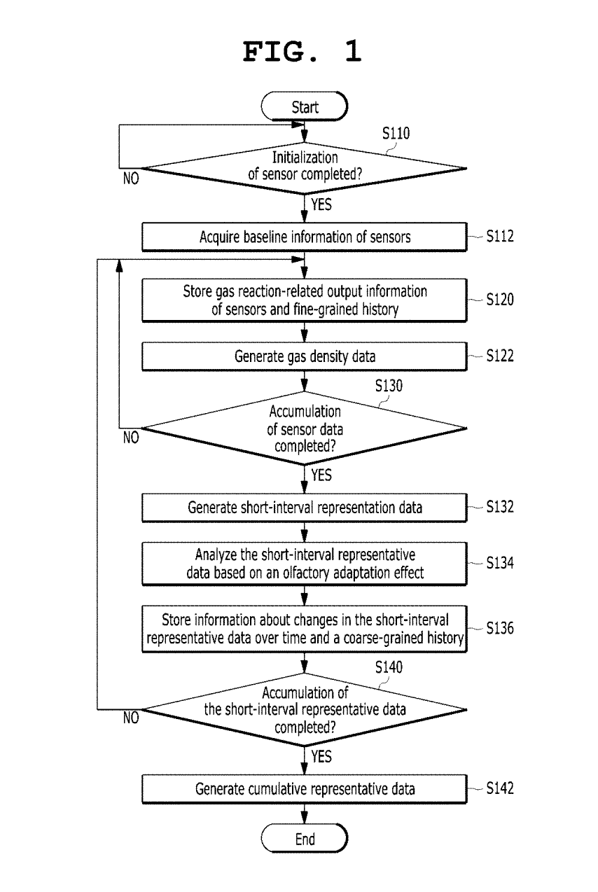

FIG. 1 is an operation flowchart showing a method for generating olfactory information, which generates representative data, including the original data of a gas density and the quantitative evaluation of a real-world odor, according to an embodiment of the present invention. Referring to FIG. 1, in the method for generating olfactory information, which generates olfactory information that may be shared between the real world and at least one virtual world, it is checked whether the initialization of the sensor has been completed at step S110.

The initialization of the sensors is repeated until the initialization of the sensor is completed. When the initialization of the sensors has been completed, the baseline information of the sensors is acquired at step S112. The baseline information refers to a measured value of a gas sensor based on a place and an environmental value in a zero-gas state, and may be subjected to a zero-point adjustment.

The gas reaction-related output information of the sensors and the fine-grained history are stored at step S120. The fine-grained history refers to the measured raw data of the sensor over a short time interval obtained by subdividing a time interval.

Gas density data is generated at step S122. It is checked whether the accumulation of the sensor data has been completed at step S130, and short-interval representative data is generated at step S132 after the accumulation of the sensor data has been completed. The short-interval representative data refers to data that is obtained by converting information collected by the sensor over a short time interval into a strength perceived by humans based on the result of an organoleptic test perceived by humans.

The short-interval representative data is analyzed based on the olfactory adaptation effect at step S134. Information about changes in the short-interval representative data over time and a coarse-grained history are stored at step S136. The coarse-grained history refers information accumulated over a time interval set to a medium or long period, and may include raw data collected by the sensors.

It is checked whether the accumulation of the short-interval representative data has been completed at step S140. After the accumulation of the short-interval representative data has been completed, cumulative representative data is generated at step S142.

The original data of the result of the detection of real-world odor by the sensors capable of recognizing a real-world odor can be acquired. In this case, quantitative information about an actually detected gas, i.e., an odor molecule, is acquired. In this case, the original data may be represented by using a chemical gas density item to be described later.

After the original data has been acquired, representative data including the evaluation of the quantitative numerical value of the real-world odor may be acquired by means of an analysis processor for the original data. In this case, the evaluation of the quantitative numerical value refers to information about the result of a judgment that is made based on the strength of the odor that can be sensed by human olfaction.

Olfactory information based on odor strength is generated by generating real-world olfactory information including the original data and the representative data acquired at the previous steps. In this case, the olfactory information refers to information having a format, such as an XML format. The olfactory information may include both the original data and the representative data. This enables a real-world mood to be more accurately conveyed when the real-world olfactory information is reconstructed in a virtual world.

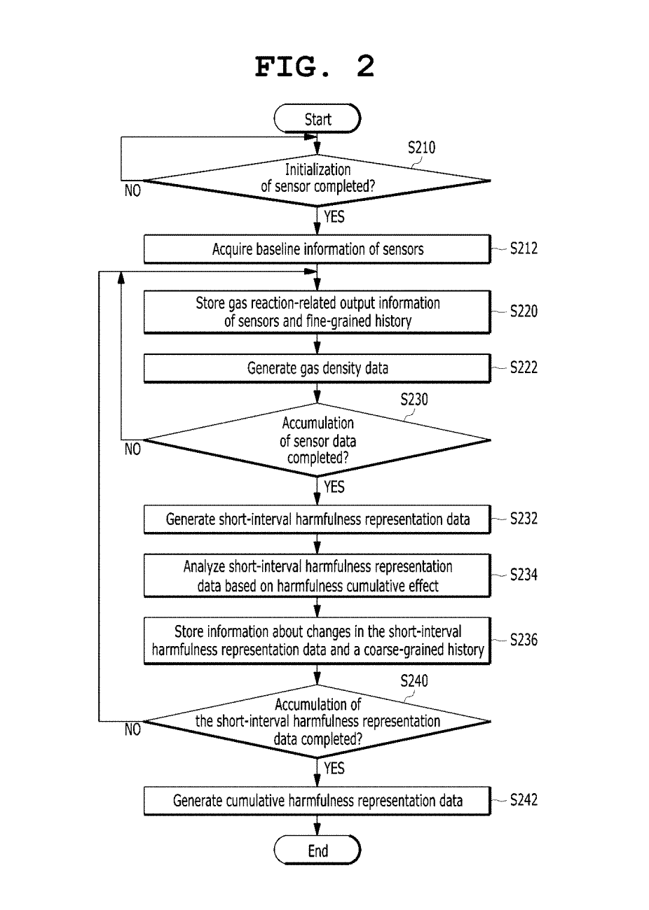

FIG. 2 is an operation flowchart showing a method for generating olfactory information, which generates representative data, including the quantitative evaluation of the harmfulness of a real-world odor, according another embodiment of the present invention. Referring to FIG. 2, in the method for generating olfactory information, which generates olfactory information that may be shared between the real world and at least one virtual world, steps S210, S212, S220, S222 and S230 of FIG. 2 are the same as steps S110, S112, S120, S122 and S130 of FIG. 1, and thus redundant descriptions will be omitted.

After the accumulation of sensor data has been completed at step S230, short-interval harmfulness representation data is generated at step S232. The short-interval harmfulness representation data refers to data that is obtained by converting information collected by the sensor over a short time interval into a quantitative evaluation value for harmfulness based on the result of the evaluation of harmfulness to humans.

The short-interval harmfulness representation data is analyzed based on a harmfulness cumulative effect at step S234. That is, when the level of harmfulness is low in the short-interval harmfulness representation data, harmfulness to a human body may be increased by long-term exposure to the human body. When a human body has been exposed to a specific gas over a long period of time, the gas may be accumulated in the human body and harmfulness may continue. Accordingly, it is necessary to analyze the short-interval harmfulness representation data based on exposure time and the harmfulness cumulative effect.

Information about changes in the short-interval harmfulness representation data over time and a coarse-grained history are stored at step S236. The coarse-grained history refers information accumulated over a time interval set to a medium or long period, and may include raw data collected by the sensors.

It is checked whether the accumulation of the short-interval harmfulness representation data has been completed at step S240. After the accumulation of the short-interval harmfulness representation data has been completed, cumulative harmfulness representation data is generated at step S242.

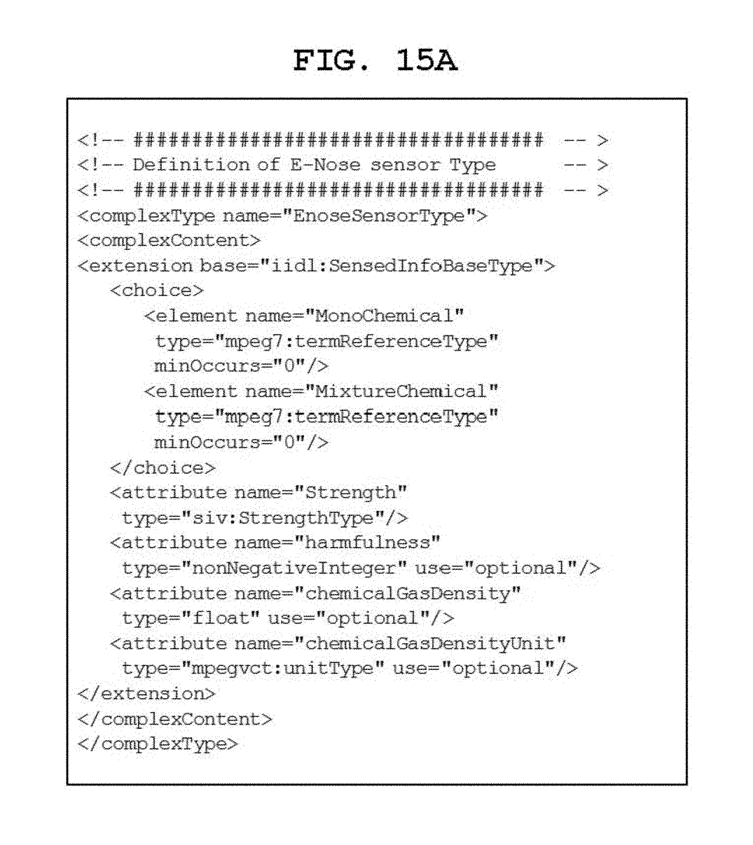

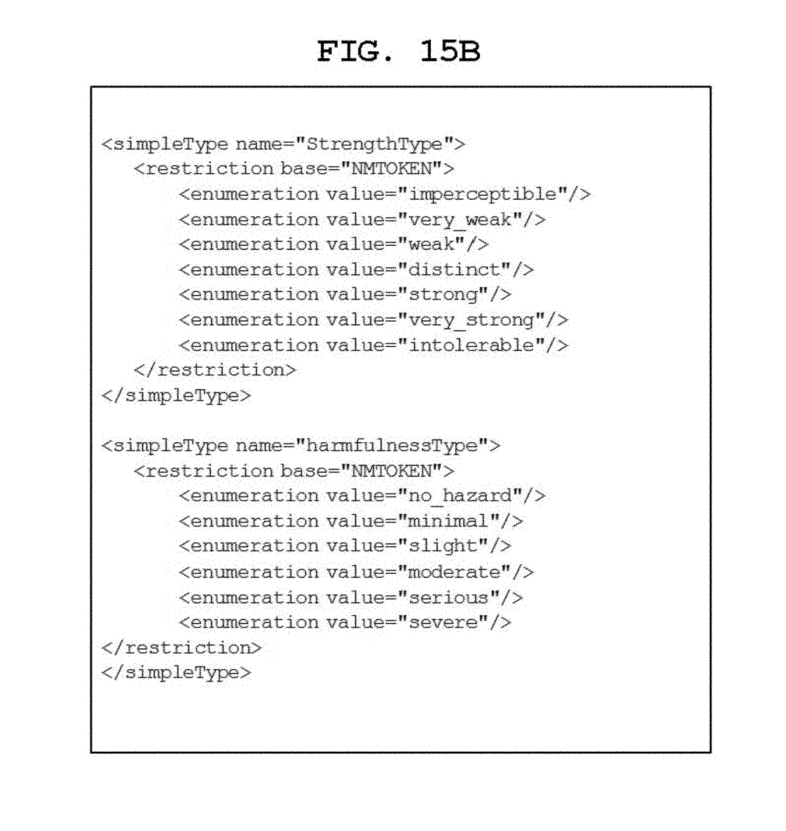

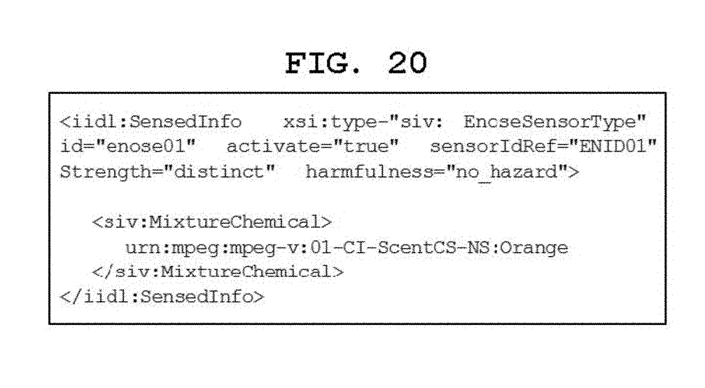

After the original data has been acquired, harmfulness representation data including the quantitative evaluation of the harmfulness of the real-world odor may be acquired by means of an analysis processor for the original data. In this case, the quantitative evaluation of the harmfulness refers to information about the result of a judgment that is made based on whether the detected odor has harmfulness. When the harmfulness cannot be represented by using only a numerical value, it may be provided in the form of the result value of evaluation. The result value of the evaluation of harmfulness that is introduced in FIG. 15 or 20 to be described later may be an example of the quantitative evaluation.

Olfactory information including information about whether the detected odor corresponds to a harmful material is generated through a process of generating real-world olfactory information including the harmfulness representation data acquired at the previous step. In this case, the olfactory information refers to information having a format, such as an XML format.

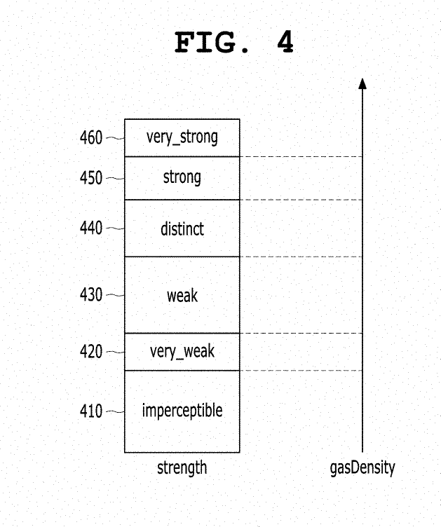

FIG. 4 is a diagram showing correlations between the strengths (gas densities) of real-world odors and quantitative evaluation indices perceived by humans according to an embodiment of the present invention.

Referring to FIG. 4, there is shown an example of density ranges 410 to 460 to which quantitative evaluation indices for specific gas densities detected by the sensor 310 may correspond.

The density range 410 is a range in which humans cannot perceive an odor. The evaluation index corresponding to the density range 410 is “imperceptible.” An evaluation index corresponding to the density range 420 is “very_weak.” An evaluation index corresponding to the density range 430 is “weak.”

An evaluation index corresponding to the density range 440 is “distinct,” an evaluation index corresponding to the density range 450 is “strong,” and an evaluation index corresponding to the density range 460 is “very_strong.” The example of the evaluation indices shown in FIG. 4 is merely an embodiment, and the spirit of the present invention is not limited by the embodiment. The ranges corresponding to the evaluation indices may be subdivided, or may be simplified. In the following, for ease of description, it is assumed that each of the density ranges 410 to 460 is represented by the median value of the corresponding density range. In particular, when real-world olfactory information is reconstructed in a virtual world, an actual gas density may be reconstructed without change in the virtual world, but virtual olfaction may be reconstructed based on the median values of the density ranges 410 to 460.

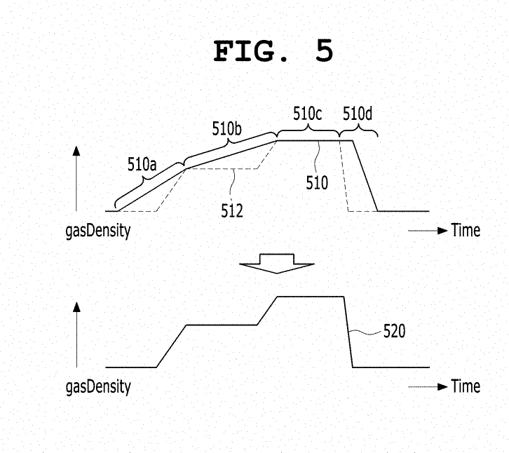

FIG. 5 is a diagram showing correlations between changes in the strength of a real-world odor over time and quantitative evaluation indices perceived by humans over time according to an embodiment of the present invention.

A curve 510 represents changes in gas density over time. In contrast, quantitative evaluation indices may be represented by a curve 512. That is, there may be differences between actual changes in gas density and gas densities (the representative values of corresponding density ranges) indicated by the evaluation indices, which correspond to differences between the curve 512 and the curve 510. As time elapses, a human may experience olfactory adaptation attributable to the fatigue of his or her olfaction and thus may not perceive an odor. When the olfactory adaptation effect is taken into account, a quantitative evaluation index may more rapidly transition to an imperceptible state even when gas density is actually maintained at a value equal to or greater than a threshold value.

In a first time interval 510a, although the curve 510 for actual gas density does not correspond to 0, the curve 512 for strength perceived by humans may correspond to “imperceptible,” i.e., 0. In a second time interval 510b, the curve 510 for actual gas density may correspond to a weak interval in the curve 512 for strength perceived by humans. In a third time interval 510c, the curve 510 for actual gas density may correspond to a distinct interval in the curve 512 for strength perceived by humans.

After the third time interval 510c has elapsed, olfactory adaptation occurs, and thus the curve 512 for the strength of the odor perceived by humans drops to 0. That is, in a fourth time interval 510d after the occurrence of the olfactory adaptation, the curve 510 for actual gas density has a value other than 0 while the curve 512 for the strength of the odor perceived by humans has a value of 0.

As described above, the curve 510 representative of actual changes in gas density may be converted into the curve 520 representative of changes in the quantitative evaluation index over time by the processor 320. In this case, the processor 320 may convert the curve 510 into the curve 520 based on mapping relationships between actual gas densities and the density ranges 410 to 460 corresponding to quantitative evaluation indices, shown in FIG. 5, and the olfactory adaptation effect.

When the changes in gas density over time, which are shown in FIG. 5, may be reconstructed in the real world without change, it may be possible not only to convey the odor and but also to reconstruct a mood conveyed by the odor without change, rather than simply conveying only the odor. For example, there may be reconstructed a mood in which an odor spreads gradually and exhibits a delicate fragrance, a mood in which an odor exhibits an explosive and strong fragrance, or a mood in which an odor spreads gradually in its initial stage, the density of the odor is explosively increased, and the odor is changed into a strong odor that reeks aloud.

A mood that is formed by an odor may be more accurately reconstructed by conveying olfactory information including both changes in gas density over time and changes in the quantitative evaluation index, as shown in FIG. 5.

In this case, when both the gas density and the quantitative evaluation index are taken into account, more elaborate reconstruction may be possible. For example, in the case where the evaluation index is in an imperceptible state, when whether the gas density is actually “0” or not is taken into account, the strength of an odor that is reconstructed in a virtual world may be set to “0” or a representative value other than “0.”

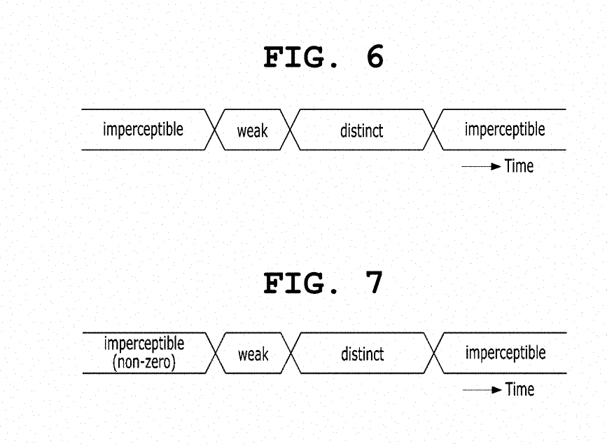

FIGS. 6 and 7 are diagrams showing the representative data of quantitative evaluation indices over time according to embodiments of the present invention.

FIG. 6 shows a case where a state sequentially transitions to an imperceptible state, a weak state, a distinct state, and an imperceptible state over time. In this case, it is difficult to distinguish whether the initial imperceptible state is completely the same as the final imperceptible state or not.

As shown in FIG. 7, an imperceptible state may be classified into and represented by an imperceptible (non-zero) state and an imperceptible (zero) state. The changes in the quantitative evaluation index shown in FIG. 7 may be implemented to correspond to the actual gas density of FIG. 4 and the quantitative evaluation index graph of FIG. 5.

The first time interval 510a of FIG. 5 corresponds to a case where an actual gas density has a value other than “0” and the value is a value less than a threshold value, i.e., a lower limit value that can be perceived by humans. In this case, a corresponding quantitative evaluation index exhibits an imperceptible state. When an actual gas density is taken into account, a compensated quantitative evaluation index in an initial state may be represented by an imperceptible (non-zero) state, like the first state of FIG. 7.

In contrast, the fourth time interval 510d of FIG. 5 includes both an interval in which an actual gas density is not “0” and an interval in which an actual gas density is “0,” and a quantitative evaluation index is represented by an imperceptible state due to the olfactory adaptation effect. In this case, in view of the olfactory adaptation effect, even when an actual gas density is not “0,” the strength of an odor perceived by humans may be considered to have an imperceptible state. That is, since a final state is recognized as an imperceptible state regardless of whether the actual gas density is “0” or not, a compensated quantitative evaluation index in the final state may be represented by an imperceptible (zero, non-zero, or don't care) state, as shown in FIG. 7.

In an embodiment, unlike in FIG. 7, the final state of FIG. 7 may be classified into and represented by an imperceptible (zero) state and an imperceptible (zero/non-zero) state. This is an embodiment to which the actual gas densities of FIG. 5 have been more accurately incorporated.

When the initial state is represented by the imperceptible (non-zero) state, the gas density of an odor reconstructed in a virtual world may be implemented using the median value of the density range 410 of FIG. 4. In contrast, an imperceptible (zero) state may be implemented as the gas density “0” of the odor. The curve 510 for actual gas density shown in FIG. 5 increases continuously in the first time interval 510a even in the imperceptible state of the curve 520, and transitions to a weak state after the threshold value. In this case, when the initial imperceptible state of the first time interval 510a is simply reconstructed as the gas density “0,” the gas density must be sharply changed upon transitioning to a weak state in the second time interval 510b, and thus it is difficult to perform control. The gas density or an odor used to reconstruct virtual olfaction in a virtual world may be implemented using an actual gas density, or may be implemented based on a quantitative evaluation index. When the gas density of an odor for the reconstruction of virtual olfaction is implemented based on quantitative evaluation indices, an imperceptible state is classified into and represented by zero, non-zero, and don't care states based on the compensated evaluation indices of FIG. 7, thereby providing an effect of effectively reconstructing target virtual olfaction while reducing the energy required for the generation of the odor.

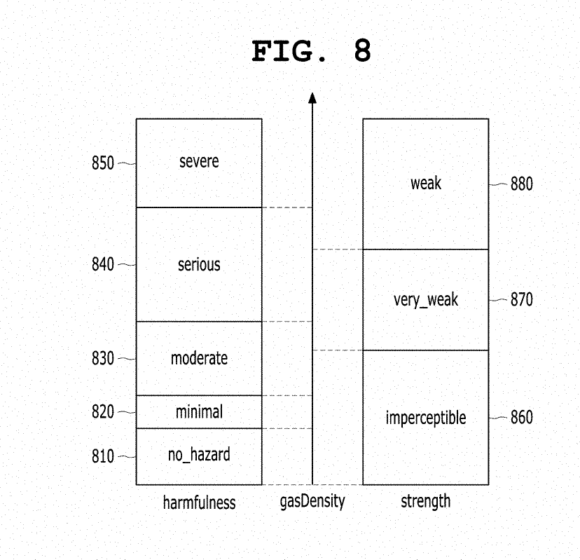

FIG. 8 is a diagram showing correlations between the strengths (gas densities) of real-world odors, quantitative evaluation indices perceived by humans, and evaluation indices for harmfulness according to an embodiment of the present invention.

A density range 860, a density range 870, and a density range 880 are reference ranges representing the strengths of odors perceived by humans in conjunction with quantitative evaluation indices. Since the density range 860, the density range 870, and the density range 880 are the same as the density ranges 410 to 430 of FIG. 4, redundant descriptions will be omitted.

The density range 810 is a range that is harmless to humans. An evaluation index corresponding to the density range 810 is “no_hazard.” An evaluation index corresponding to the density range 820 is “minimal,” and an evaluation index corresponding to the density range 830 is “moderate.” An evaluation index corresponding to the density range 840 is “serious,” and an evaluation index corresponding to the density range 850 is “severe.”

The example of the evaluation indices shown in FIG. 8 is merely an embodiment, and the spirit of the present invention is not limited by the embodiment. The ranges corresponding to the evaluation indices may be further subdivided, or may be simplified.

When an actual gas density, a quantitative evaluation index perceived by humans, and an evaluation index for harmfulness are inconsistent with one another, a safety problem may occur. For example, in an imperceptible density range 860 that is lower than a threshold value, i.e., a lower limit perceived by humans, as shown in FIG. 8, the evaluation index for harmfulness may change from “no_hazard” through “minimal” to “moderate.” That is, when a human has been exposed to a harmful gas in a density range 820 having minimal harmfulness over a long period of time without perception, or when a human has been exposed to a harmful gas in a density range 830 having moderate harmfulness over a long period of time without perception, the human may inhale the harmful gas without perception. When a human has been exposed to a harmful gas over a long period of time as described above, there is a possibility that the gas is accumulated in a human body, the ranges of the evaluation indices of FIG. 8 may be adjusted to higher evaluation values for harmfulness when the time over which a human has been exposed to a gas increases.

Accordingly, in the present invention, based on mapping relationships among actual gas densities, quantitative evaluation indices perceived by humans, and evaluation indices for harmfulness, when a harmful gas is detected in the state in which a human cannot perceive the gas, safety can be increased by providing notification of the detection of the harmful gas. A full preparation for safety management may be made by adjusting an evaluation value for the harmfulness of a gas through the consideration of the time over which a human has been exposed to the gas.

Of gases, there is a gas that is colorless and odorless and has serious or severe harmfulness in the state in which a human does not perceive the gas, which is more serious than the case of FIG. 8. When it is determined that a harmful gas has been generated in the state in which a human cannot perceive the gas, the apparatus for generating olfactory information according to the present invention or a server connected to the apparatus may call the attention of surrounding humans and allow required measures to be taken for ensuring of safety by issuing a special alarm.

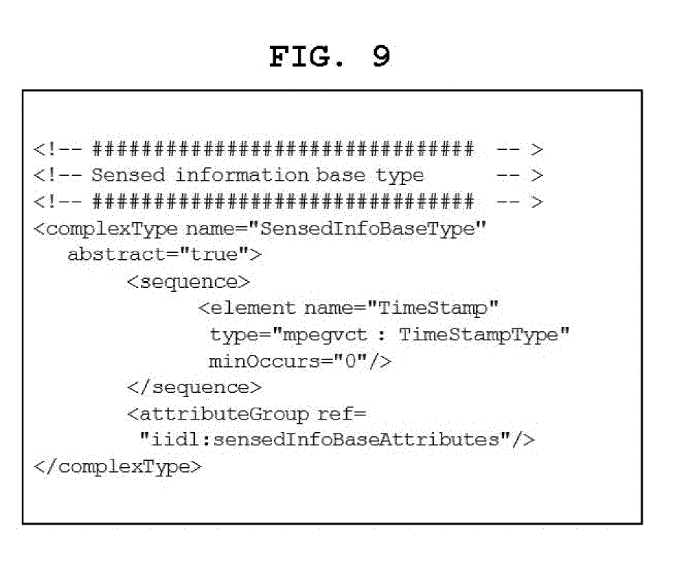

FIG. 9 is a diagram showing the XML representation syntax of Sensed Information Base Type according to an embodiment of the present invention.

To reconstruct information detected or acquired by an e-nose sensor in a virtual world or the real world so that the olfactory organ of a human body can perceive the information, a TimeStamp item indicative of the time at which the information was detected or acquired by the e-nose sensor is introduced. In view of the characteristics of the olfactory organ in which the olfactory organ is more sensitive and more easily becomes fatigued than other organs, it is significantly important to include a time-related item in olfactory information. TimeStamp may be defined based on the provision “there is a choice of selection among three timing schemes, which are absolute time, clocktick time, and delta of clock tick time” stipulated in the ISO/IEC 23005-6 standard.

TimeStamp is information that is used to stipulate information detected or acquired by an e-nose sensor. As shown in FIG. 9, TimeStamp may be defined as a sub-item of SensedInfoBaseType, i.e., a type of SensedInfoType. FIG. 9 shows an XML representation syntax in which TimeStamp is defined as a sub-item of SensedInfoBaseType.

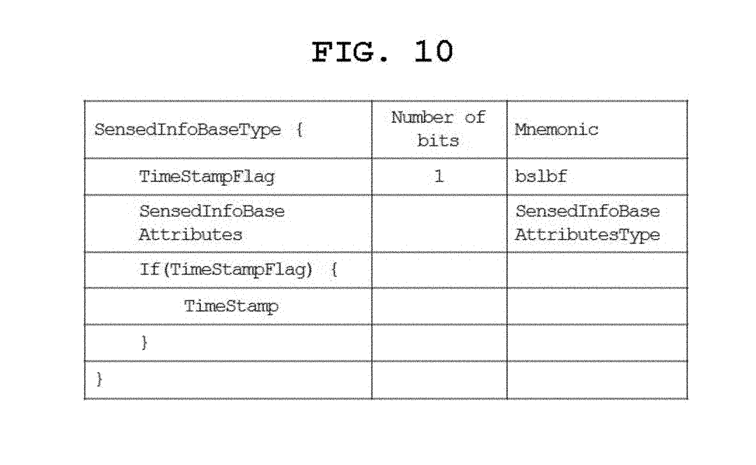

FIG. 10 is a diagram showing the binary representation syntax of SensedInfoBaseType according to an embodiment of the present invention. Referring to FIG. 10, the binary representation syntax of SensedInfoBaseType can be seen.

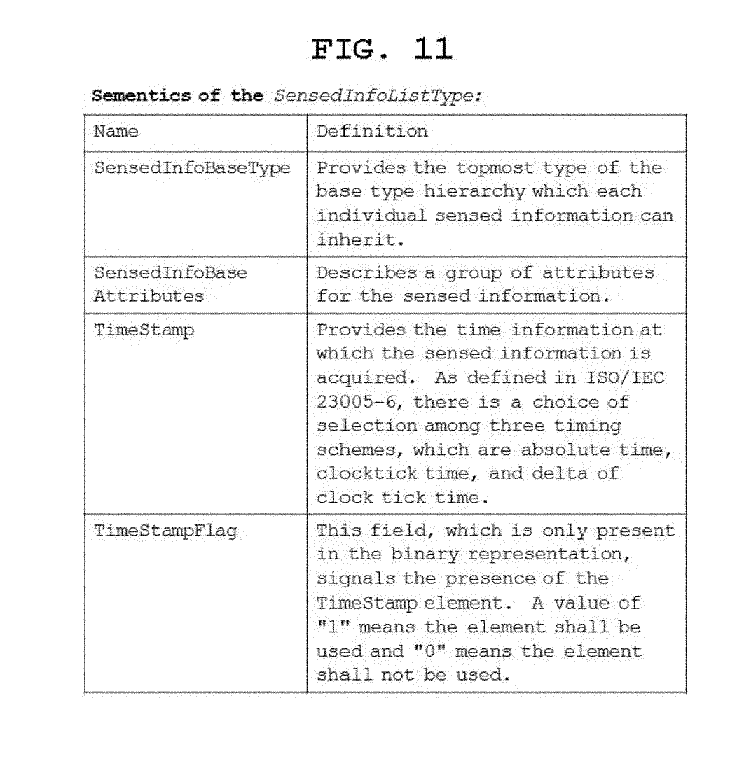

FIG. 11 is a diagram showing the semantics of SensedInfoListType according to an embodiment of the present invention. Referring to FIG. 8, the details of SensedInfoListType based on the semantics can be seen. The TimeStamp item provides information about the time at which information detected by an e-nose was acquired. The TimeStamp item may be represented by absolute time, time clocktick time, or delta of clock tick time, as described above. A TimeStampFlag item is a flag indicative of whether a TimeStamp item is present or not.

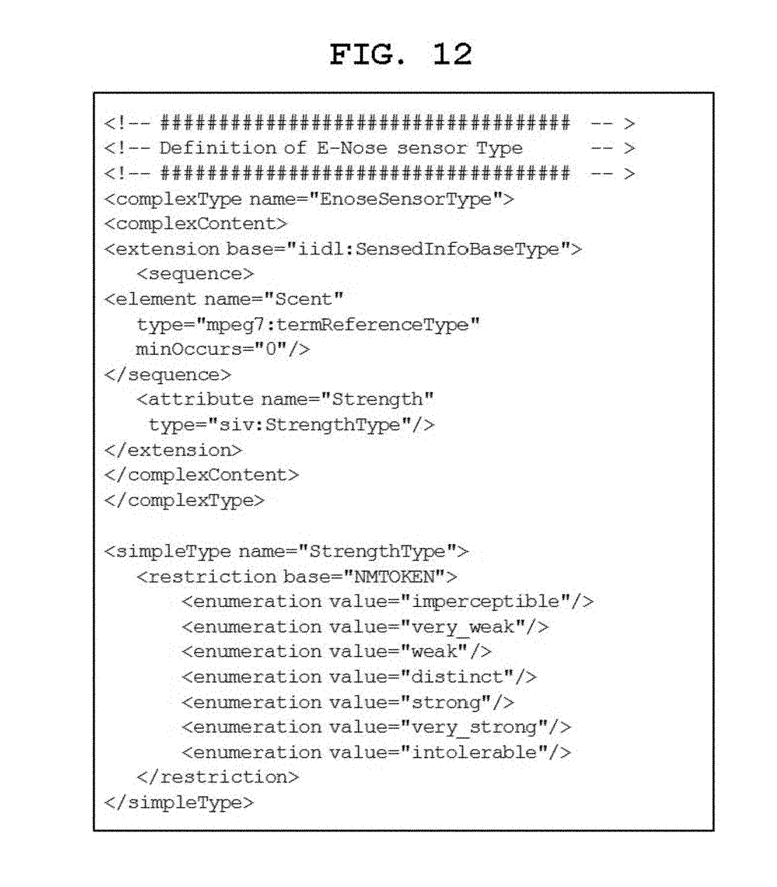

FIG. 12 is a diagram showing the binary representation syntax of the strengths of odors according to an embodiment of the present invention. The strengths shown in FIG. 12 refer to strength values that are estimated to be actually perceived by humans from the strengths of odors detected by an e-nose. Since humans have olfactory adaptation, olfaction becomes fatigued when they have been exposed to the same odor over a predetermined period or more or at a predetermined strength or more, and thus they cannot perceive the odor any longer or the strength of the odor that can be perceived by them may be limited. The strengths shown in FIG. 12 may be values into which estimations based on the olfactory adaptation phenomenon have been incorporated. A process of mapping the strengths of odors, actually detected through organoleptic tests, to the strengths of odors, such as those shown in FIG. 4 may be added.

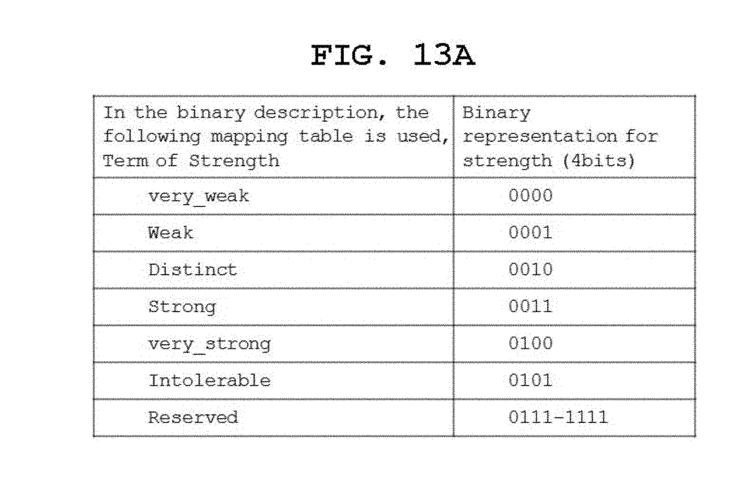

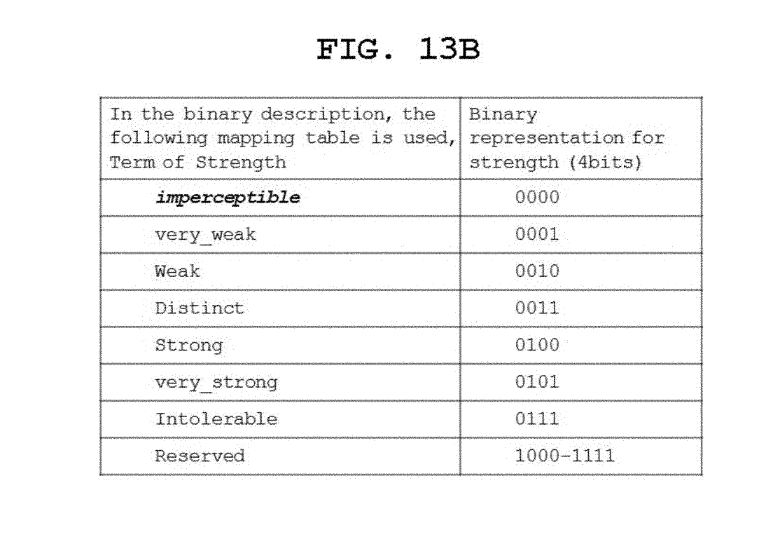

Referring to FIG. 13A, the binary representation syntax of the strengths of odors can be seen. In this case, the strength of an odor may be represented by four bits. “0000” refers to “very_weak,” “0001” refers to “weak,” “0010” refers to “distinct,” “0011” refers to “strong,” “0100” refers to “very_strong,” “0101” refers to “intolerable,” and “0111-1111” may be reserved for future use.

FIG. 13B is a diagram showing the modified strength binary representation syntax of the strengths of odors in which an imperceptible step has been added to the binary representations of the strengths of odors according to an embodiment of the present invention.

There is an odor for which the density of particles is not 0 according to the result of the detection by an e-nose and which cannot be perceived by an olfactory organ. Accordingly, to more realistically represent the strength perceived by an olfactory organ, the imperceptible step is added. Therefore, each of the existing binary representations of the strengths shown FIG. 13A is increased by one in FIG. 13B due to the introduction of the imperceptible strength.

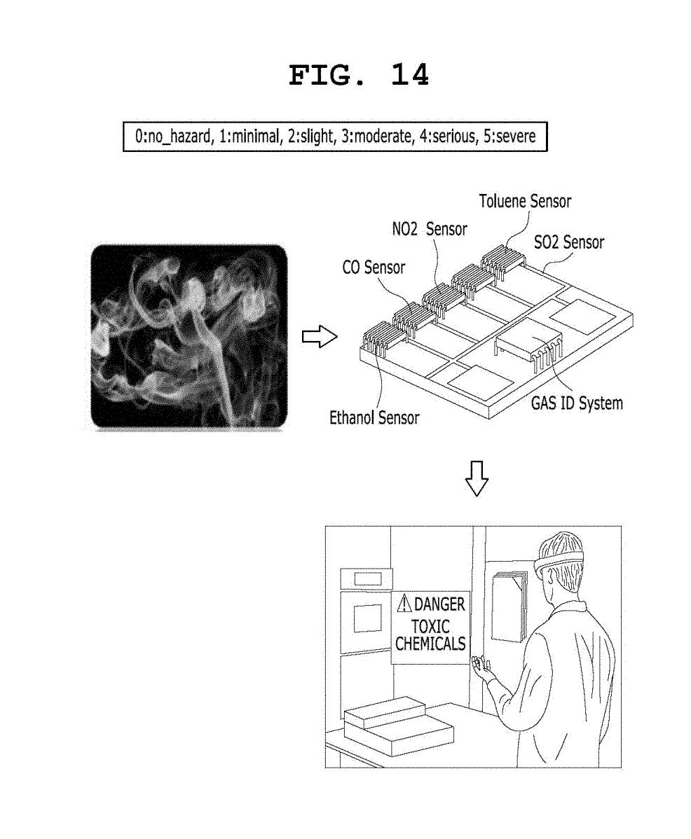

FIG. 14 is a diagram schematically showing a process of generating information about the generation of a hazardous odor when the hazardous odor is generated during a process of generating olfactory information according to an embodiment of the present invention. As can be seen from the left upper end of this diagram, detected odors may be classified into six steps ranging from no hazard (grade 0) to a serious level (grade 5). FIG. 14 schematically shows a use case of matching an odor detected by an e-nose against odor information previously stored in an odor database and then evacuating people to a safe location when it is determined that a hazardous odor has been generated. However, when this configuration is used, it is difficult to accurately grade all odors due to the diversity of the molecules of odors all over the world.

FIGS. 15A and 15B are diagrams showing the XML representation syntax of E-Nose Sensor Type according to an embodiment of the present invention. Referring to FIG. 15, the XML representation syntax of E-Nose Sensor Type can be seen.

Referring to FIG. 15A, “strengthType” and “harmfulnessType” are introduced as sub-items of SensedInfoBaseType, and a “chemicalGasDensity” item is also introduced in the middle of an attribute name. In this case, “chemicalGasDensity” refers the density of a gas actually detected by an e-nose, and “chemicalGasDensityUnit” may be given, for example, in ppm. “strengthType” refers a value that is obtained through conversion into or estimation of the strength that can be perceived by humans. Accordingly, the numerical value of “strengthType” may be provided in a form in which the numerical value is expressed in ppm and into which the evaluation value of a sensory organ has been incorporated. In FIG. 15B, “imperceptible”, “very_weak”, “weak”, “distinct”, “strong”, “very_strong”, and “intolerable” which are shown as examples of the values of “strengthType” are examples of values into which the evaluation values of a sensory organ have been incorporated.

As to “harmfulness,” the result value of the evaluation of harmfulness may be provided in addition to a numerical value. In FIG. 15B, “no_hazard”, “minimal”, “slight”, “moderate”, “serious”, and “severe,” which are shown as examples of the values of “harmfulness,” may be viewed as examples of the result values of the evaluation of harmfulness.

Of harmful gases, there may be gases that are classified as colorless and odorless gases by humans but are extremely harmful. Accordingly, when an e-nose is used to diagnose the safety of a factory, a use case using both “strengthType” and “harmfulness” may be significantly advantageously utilized.

When the real-world olfactory information detected and generated by an e-nose is shared in the real world or a virtual world, it may be significantly useful to convey a corresponding mood without change. Accordingly, it will be significantly effective in sharing olfactory information between a virtual world and the real world to provide both a “chemicalGasDensity” value actually detected by an e-nose and “strengthType” obtained by reevaluating the strength of an odor based on human olfaction.

When real-world olfactory information is shared in a virtual world, the information about the time for which a human has been exposed to an odor may be also advantageously used. That is, when the TimeStamp item and the “stengthType” item previously introduced in FIGS. 9 to 11 are provided together, a mood related to an odor actually detected by an e-nose may be easily reconstructed in a virtual world.

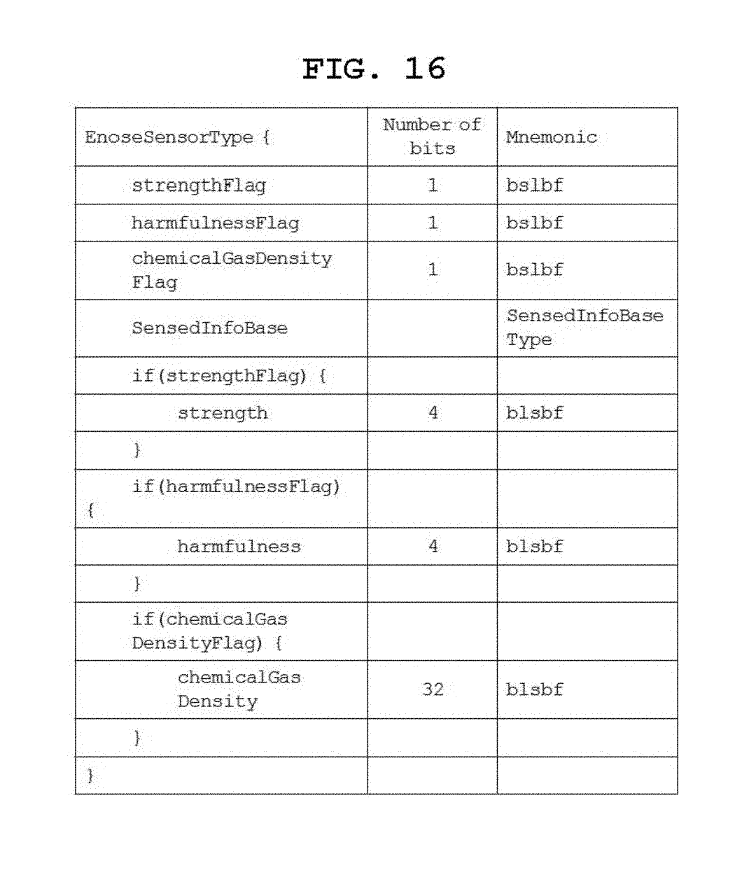

FIG. 16 is a diagram showing the binary representation syntax of Enose Sensor Type according to an embodiment of the present invention. Referring to FIG. 16, the binary representation syntax of Enose Sensor Type can be seen.

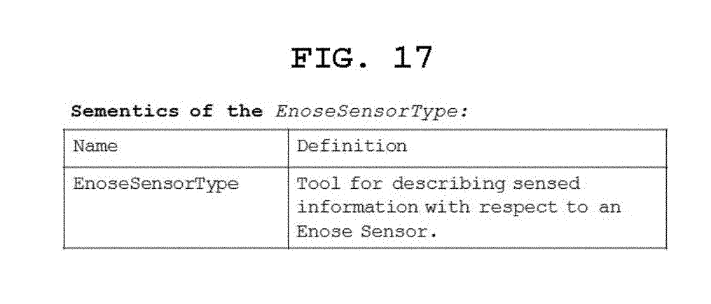

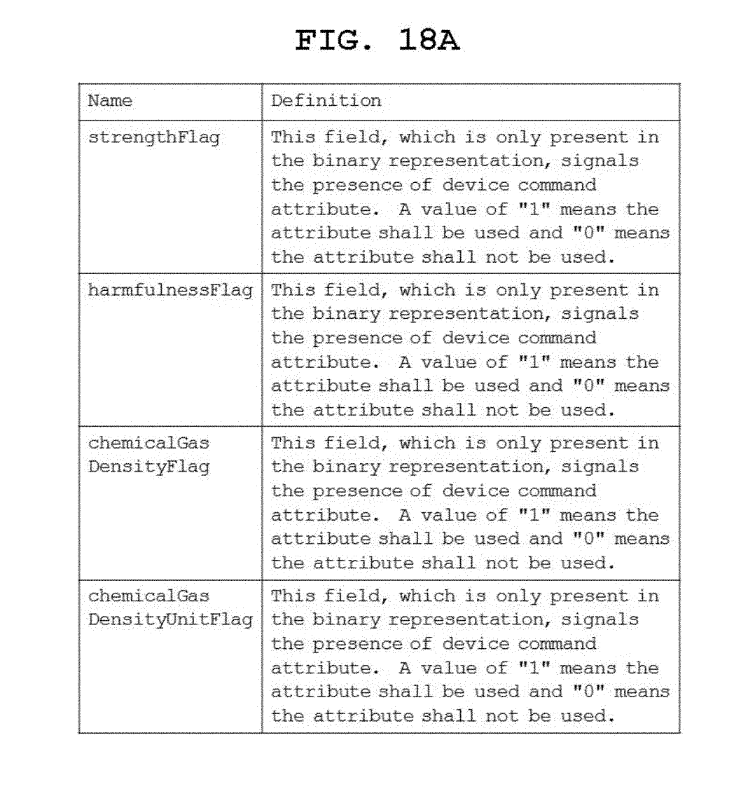

FIGS. 17, 18A, 18B, and 19 are diagrams showing the definitions of the sub-items of Enose Sensor Type according to an embodiment of the present invention. In FIGS. 17, 18A, 18B, and 19, although EnoseSensorType may define the physical sensor type of an e-nose, examples that all e-nose-related information including detected information may be included is introduced.

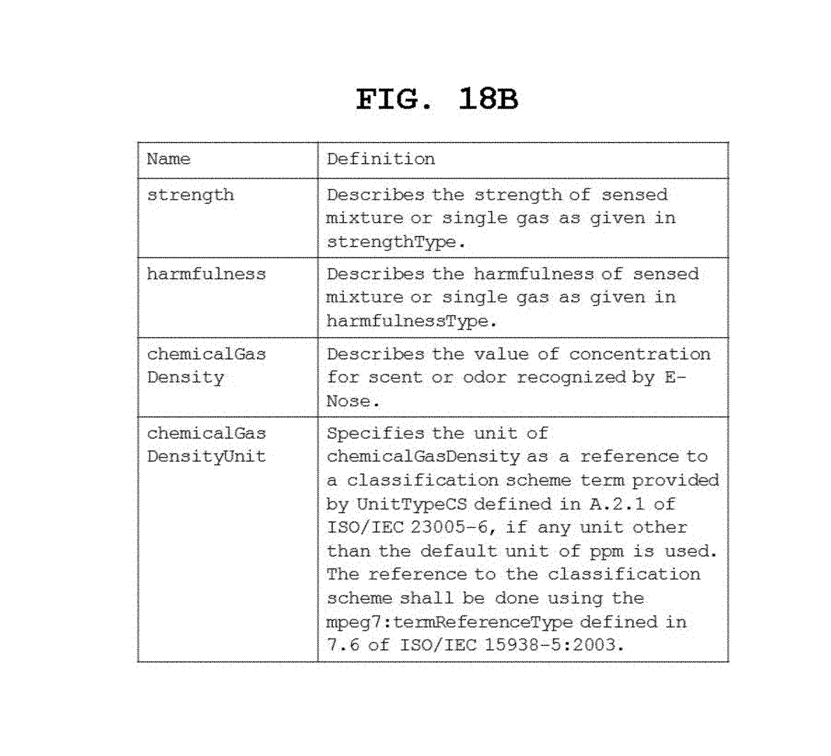

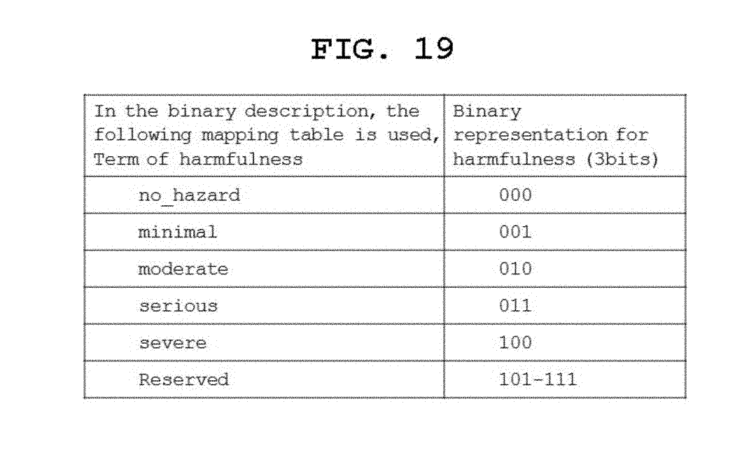

In FIGS. 18A and 18B, items, such as strength, harmfulness, chemicalGasDensity, chemicalGasDensityUnit, etc., are defined. In FIG. 19, examples of the values that the harmfulness items may have are introduced.

FIG. 20 is a diagram showing a syntax designed to define harmfulness in the XML representation syntax of E-Nose Sensor Type according to an embodiment of the present invention. Referring to FIG. 20, the XML representation syntax designed to define harmfulness in E-Nose Sensor Type can be seen.

The method according to an embodiment of the present invention may be implemented in the form of program instructions that can be executed by a variety of computer means, and may be stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

According to the present invention, intercompapatibility between a virtual world and the real world can be provided by recognizing odors present in the real world within the scope of MPEG-V and conveying the real-world odors to the virtual world.

The present invention is configured to digitize the types of odors perceived by actual olfaction, the times required for perception, and the fatigue levels of the olfactory organ of a human body in accordance with the operation of the olfactory organ of the actual human body and to then provide representations. This can contribute to the commercialization of research into the digitization of the five senses of humans, such as virtual reality, a scent display, etc.

According to the present invention, detailed information can be generated and conveyed in a process of conveying a real-world odor to a virtual world. That is, according to the present invention, the density of a gas, i.e., the cause of a real-world odor, can be conveyed to a virtual world without any change to raw data and also a quantitative evaluation result based on a human organoleptic test can be conveyed to the virtual world, thereby more faithfully reconstructing a mood related to the real-world odor in the virtual world.

According to the present invention, time-series virtual olfactory information can be included using changes in real-world gas density and changes in the strength of an odor perceived by humans over time, and a mood related to a real-world odor can be reconstructed in a virtual world without change.

According to the present invention, a method for effectively implementing virtual olfaction in a virtual world can be provided by generating representative data through the consideration of the olfactory adaptation effect for a real-world odor. Furthermore, by reducing the fatigue of human olfaction, the sensitivity of olfaction can be prevented from being reduced during the successive generation of virtual olfaction, and an olfaction conveyance effect can be increased.

According to the present invention, a full preparation for the safety management of harmfulness gases that cannot be perceived by humans can be made in such a way as to map the strength of an odor, perceived by humans for a specific gas, to a quantitative evaluation result for the harmfulness of the gas.

When gas information is extracted using a conventional gas sensor, the extraction is highly influenced by a surrounding situation and sensitivity is limited, and thus it is difficult to extract gas information having a low density regardless of the influence of a surrounding environment, with the result that it is barely conceivable that extraction using the conventional gas sensor can replace a human organoleptic test. However, low-density information can be detected due to the development of a nano-sensor and measurement can be standardized via the automation of time-series feature extraction in a complex situation due to the development of convolution neural networks, and thus this can replace a human organoleptic test.

While the present invention has been described in conjunction with the limited embodiments and drawings above, various modifications and variations can be made based on the foregoing description by those having ordinary knowledge in the art to which the present invention pertains. For example, although the described technologies are performed in sequence different from the described sequence, the described components, such as structures, devices, circuits, units, parts, and the like, are coupled or combined in a form different from the described form, and/or one or more of the components are replaced with other components or equivalents, appropriate results may be achieved.

Therefore, other implementations, other embodiments and equivalents to the claims fall within the scope of the following claims.

What is claimed is:

1. A method for generating olfactory information, which generates olfactory information that can be shared between a real world and at least one virtual world, the method comprising:
acquiring, by a sensor capable of detecting a real-world odor, original data of a result of detection of the real-world odor;
acquiring, by an analysis processor for the original data, representative data including an evaluation of a quantitative numerical value of the real-world odor, and
generating real-world olfactory information including both the original data and the representative data.

2. The method of claim 1, wherein the representative data includes an interval quantitatively representative of an imperceptible case that cannot be perceived by humans.

3. The method of claim 2, further comprising detecting a situation, in which a specific gas cannot be perceived by the humans in the real world and presence of a component of the specific gas has been detected, by including both the original data and the representative data.

4. The method of claim 3, further comprising detecting whether the component of the specific gas, the presence of which has been detected, is harmful to a human body.

5. The method of claim 1, wherein:
the original data includes a series of results of detection of the real-world odor over time, together with timestamps; and
the representative data includes a series of evaluations of quantitative numerical values of the real-world odor over time, together with the timestamps.

6. The method of claim 5, further comprising generating a series of pieces of virtual olfactory information to be reconstructed in the virtual world by using the timestamps and also using a series of pieces of real-world olfactory information over time including the series of results of detection of the real-world odor over time and the series of evaluations of quantitative numerical values of the real-world odor over time.

7. The method of claim 6, wherein the generating a series of pieces of virtual olfactory information comprises incorporating a mood, provided by the real-world odor, into the series of pieces of virtual olfactory information to be reconstructed in the virtual world.

8. The method of claim 6, wherein the generating a series of pieces of virtual olfactory information comprises:
compensating the representative data by taking into account an olfactory adaptation effect for the real-world odor; and
generating the series of pieces of virtual olfactory information to be reconstructed in the virtual world based on the compensated representative data.

9. A method for generating olfactory information, which generates olfactory information that can be shared between a real world and at least one virtual world, the method comprising:
acquiring, by a sensor capable of detecting a real-world odor, original data of a result of detection of the real-world odor;
acquiring, by an analysis processor for the original data, first representative data including a quantitative evaluation of harmfulness of the real-world odor; and
generating real-world olfactory information including the first representative data.

10. The method of claim 9, further comprising:
acquiring, by an analysis processor for the original data, second representative data including an evaluation of a quantitative numerical value of the real-world odor, and
including the second representative data, together with the first representative data, in the real-world olfactory information.

11. The method of claim 10, further comprising detecting a situation, in which a component of a specific gas having harmfulness has been detected in the real world and the specific gas cannot be perceived by humans, by using the original data, the first representative data, and the second representative data.

12. The method of claim 9, wherein the acquiring first representative data comprises:
matching the quantitative evaluation of harmfulness of the real-world odor against preset density ranges of the real-world odor; and
acquiring a density range of the real-world odor matched to the quantitative evaluation as part of the first representative data.

13. An apparatus for generating olfactory information, which generates olfactory information that can be shared between a real world and at least one virtual world, the apparatus comprising:
a sensor configured to:
recognize a real-world odor; and
acquire original data of a result of detection of the real-world odor, and
a processor configured to:
acquire representative data including an evaluation of a quantitative numerical value of the real-world odor by analyzing the original data; and
generate real-world olfactory information including both the original data and the representative data.

14. The apparatus of claim 13, wherein the processor is further configured to include information about an interval, quantitatively representative of an imperceptible case that cannot be perceived by humans, in the representative data.

15. The apparatus of claim 13, wherein:
the sensor is further configured to generate a series of results of detection of the real-world odor over time, together with timestamps, as the original data; and
the processor is further configured to generate a series of evaluations of quantitative numerical values of the real-world odor over time, together with the timestamps, as the representative data.

16. An apparatus for generating olfactory information, which generates olfactory information that can be shared between a real world and at least one virtual world, the apparatus comprising:
a sensor configured to:
recognize a real-world odor; and
acquire original data of a result of detection of the real-world odor, and
a processor configured to:
acquire first representative data including a quantitative evaluation of harmfulness of the real-world odor by analyzing the original data; and
generate real-world olfactory information including the first representative data.

17. The apparatus of claim 16, wherein the processor is further configured to:
generate an evaluation of a quantitative numerical value of the real-world odor as second representative data by analyzing the original data; and
generate the real-world olfactory information including both the first representative data and the second representative data.
